(19) United States
(12) Patent Application Publication
(10) Pub. No.: US 2005/0233004 A1
(43) Pub. Date: Oct. 20, 2005

(54) Composition of Chinese drugs having neuro-protecting activity

(75) Inventors: Joon Shik Shin, Seoul (KR); Yeong-Ok Kim, Seoul (KR)

Correspondence Address: BIRCH STEWART KOLASCH & BIRCH, PO BOX 747, FALLS CHURCH, VA, 22040-0747, US

(73) Assignee: Yeong-Ok KIM

(21) Appl. No.: 11/108,744

(22) Filed: Apr. 19, 2005

(30) Foreign Application Priority Data
Apr. 20, 2004 (KR) 10-2004-0027034

(51) Int. Cl. A61K 35/78; A61K 35/32

(52) U.S. Cl. 424/549; 424/725; 424/757; 424/769; 424/773

(57) ABSTRACT

The present invention is to provide a composition comprising of natural products of 5˜20 weight parts of Astragali Radix, 5˜10 weight parts of Atractyodis Rhizoma, 5˜10 weight parts of Notopterygii Rhizoma, 5˜10 weight parts of Langanae Cortex, 5˜10 weight parts of Lycii Fructus, 2˜10 weight parts of Cnidium Rhizoma, 2˜10 weight parts of Glycyrrhizae Radix, 2˜10 weight parts of Horn of Cervi Parvum, 5˜10 weight parts of Polygoni Multiflori Radix, 2˜10 weight parts of Paeonia Radix of which substances are powdered or extracted with water, alcohol or mixture thereof and the present composition has excellent neuro-protecting activity.

### TECHNICAL FIELD

The present invention relates to a composition of Chinese drugs (natural products) having neuro-protecting activity.

### BACKGROUND ART

A dementia can be mainly divided into Alzheimer's disease (About 60-70% of total dementia), bloody dementia, dementia of special brain disease or dementia of systemic disease.

A representative Alzheimer's disease is known as accumulation of beta-amyloid in brain and neurotoxin are main factors (Selkoe D J. Normal and abnormal biology of the beta-amyloid precursor protein. Annu rev Neurosci b 1994; 17: 489-517) and is a disease of which region that donates judgment, memory and speech function in brain is injured. This disease has an important rate in USA and a study and development for therapeutic and preventive agent against the disease are being actively carried out. However, developed therapeutics are effective only in early stage of disease. Therapy is aimed mainly for supplement of deficient neurotransmitter such as choline series and monoamine series or for metabolic accentuation of remaining neurons. Progressive effect of awareness in Alzheimer's disease is only confirmed in some studies.

An onset of a Vascular dementia (about 20-25% of total dementia) is originated from repeated cerebral apoplexies and brain injuries therefrom. Though it is easy to be thought that a cerebral apoplexy which brings about chronic somatic disorders such as hemiplegia or speech disorder and in case repeated, cerebral injuries are combined and finally, a dementia arose. In Korea, as dementia patent is apt to rely on folk medicine and does not continually control cerebral diseases such as blood pressure, diabetes, dementia problem is much more problem than in USA. In cerebral ischemia model, the content of acetylcholine which executes learning and memory in hippocampus is remarkably lower. This is regarded that parasympathetic nerve is functionally disordered due to blood flow disorder in brain. (Selkoe D J. Normal and abnormal biology of the beta-amyloid precursor protein. Annu rev. Neurosci 1994; 17: 489-517; Ni, J. W. Ohta, H., Matsumoto, K. Watanabe, H.; Progressive cognitive impairment following chronic cerebral hypoperfusion induced by permanent occlusion of bilaterral carotid arteries in rats. Brain res. 653, 213(1994)).

A dementia due to specific brain disease and systemic disease may be originated from rare kinds of degenerative brain disease, Parkinson's syndrome, chicken pox, cerebral hemorrhage by brain injury and brain tumor. As most of them progress gradually, it is apt to be misunderstood as Alzheimer's disease. As another occasion, malignant anemia, chronic hepatic disease, functional disturbance of thyroid, syphilis, vasculitis etc. are also to be occasions of dementia.

As for symptom of dementia, first anamnesis is mainly appeared, in case this symptom is repeated, learning of new information and reservation thereof are impossible and therefore, learning power is strongly lowered. As time passes through, memory power is gradually lowered and at last does not memorize very earlier thing. Second, trouble of space-perception power is appeared. A patent loses its way even though a familiar place. In severe case, the patent can not find rest room or lavatory. At a glance, as the patent seems to be visual disorder due to disorder of optic nerve or retina, such symptom is, in fact, a dysfunction due to brain lesion, specially dysfunction of parietal lobe cortex. Third. Disorder of judgment is appeared. The disorder of judgment means that the patent can properly cope with various problems of surroundings.

A brain stroke appears with locomotive dysfunction and perception dysfunction such as dysfunctions of working memory, consolidation or susceptibility to interference.

Anatomical tissues related with memory are hippocampus, cortex, mamillary body, thalamic nuclei, amygdala and ascending reticular formation. The amygdala and hippocampus relate to short time memory and temple cortex passing through temple and serve fiber connecting caudate nucleus and pulvinar. (M. S. Park; Psycobiology, Seoul, 222-235, 1994, Jishiksanupsa Publ. Co.). However, memory is not an independent activity of any special region but many nerve circuits communicate one another and various changes occur according to stimuli of new learning. (C. W. Lee, J. H. Lee, Brain and mentality, Seoul, Gyoyukkwahaksa Publ. Co. 421-423, 1989).

Learning can be defined as “relatively perpetual change of behavior arose.” (J. Yoon, I. Sung, Old man Psychology, Seoul Jungangjuksung Publ. Co., 421-423; D. S. Hong, Outline of Psychology, Seoul, Bakyungsa Publ. Co., p 143-144, 1985). Learning is a alteration in an organic tissue created through experience. This alteration renders influence on activity of an organism and Long lasting of the alteration refers to as memory and also refers to as “ability of using the memory, in case of necessity. (H. K. Lee, Learning Psychology, Seoul, Tamkudang Publ. Co., p 7, 1990). Learning and memory let neurotransmitters alterate morphologically in synapses and let content of acetylcholine in central choline neurotransmitting series. Learning and memory are fields of studies through mouse test.

In the selection of animal model for disease of central nervous system, test animals impaired hippocampus due to apoplexy show dysfunctions of ability learning of space memory in Morris' water maze test (Chrobak J J. Hanin I, Wassh T J. Af63A (ethylcholine aziridinum ion), a cholinergic neurotoxic, selectively impairs working memory in a multiple component T-maze task, Brain Research. 414; 15-21, 1987). Acetylcholine in brain of Alzheimer patent is exhausted. It is reported that scopolamine, an anticholinergic agent induces loss of memory, similar to Alzheimer's disease. It is reported that scopolamine suppresses passive conditional avoidance response. There are no reports on effect influenced to active conditional avoidance response. (Alan, H. (1993). Neurotoxins as tools in lesioning experiments in natural and synthetic neurotoxins (Academic Press Inc., San Iiegok USA), p. 1-46; Craig P. S., Gina, M. B., Wayne, P., Mary, L., David, E. S. and Douglas, K. R. (1997) Pharmacological activity and safety profile of P10358, a novel, orally active acetyltransferase inhibitor for Alzheimer's disease. J. Pharmacol. Exp. Thr., 280. 710-720.)

Especially, hippocampus and frontal cortex are regions which perform an import role in memory and perception in Alzheimer's disease. In case these impair, arise a severe dysfunction on learning Morris's water maze space memory. Water maze is a pool which was filled with opaque water and a platform is hided at the very below of water so as for mice to find platform hided under the water and escape. This model is easy to control other clues than space information. In the test circumstances, within configuration of the clues which can be utilizable, mice should learn their positions, that is, by using distal cud in the maze, and should find platform hided. To solve such problems, it says that memory of spacial reference is necessary.

Among therapeutic agents known until now, galantamine has double activities which blocks not only action of acetylcholinesterase but also intensifies the activity of acetylcholine in nicotine receptor. Taclin has severe hepatotoxicity, administration method is difficult and has low activity as therapeutics. Donepezil selectively which acts on acetylcholinesterase, is metabolized in liver and is excreted in kidney. Donepezil was approved as therapeutics from FDA in USA November, 1996 and in Korea it was approved as aricept(tradename) from KFDA. Huperzia A which was distributed as Qian Ceng Ta in China blocks acetylcholinesterase and this drug chemically binds with active site and blocks decomposition of the enzyme by confirming X-ray crystallography.

### SUMMARY OF THE INVENTION

The present inventors carried out an intensive study and found out the fact that the present composition prevents necrosis of neurons.

Accordingly, an object of the present invention is to provide a composition of comprising 5˜20 weight parts of Astragali Radix, 5˜10 weight parts of Atractyodis Rhizoma, 5˜10 weight parts of Notopterygii Rhizoma, 5˜10 weight parts of Langanae Cortex, 5˜10 weight parts of Lycii Fructus, 2˜10 weight parts of Cnidium Rhizoma, 2˜10 weight parts of Glycyrrhizae Radix, 2˜10 weight parts of Horn of Cervi Parvum, 5˜10 weight parts of Polygoni Multiflori Radix and 2˜10 weight parts of Paeonia Radix as main ingredients.

The present invention is further to provide a composition comprising one or more components selected from the group consisting of Ginseng Radix, Angelicae Gigantis Radix, Comus Fructus, Dioscoreae Rhizoma, Rehmanniae Radix, Cuscutae Semen, Shizandra Semen, Rubi Fructus, Puerariae Radix, Artemisiae Capillaris Herba, Zizyphi semen, Zingiberis Rhizoma, Cinnamomi Cortex and Muschus as auxiliary components in addition to the said main ingredients.

The present composition has nearly not side effects and toxicities and has an improved pharmacological effects comparing with ordinary therapeutics for dementia until now.

Therefore, the present invention is further to provide functional food composition comprising the main ingredients and if necessary, together with one or more components selected from auxiliary ingredients.

### BRIEF DESCRIPTION OF THE DRAWINGS

FIG. 1 is photographs of cresyl violet-stained hippocampal regions of rats by microscope. In FIG. 1, A and B are photographs of coronal slices of dorsal hippocampus of rats of control group (sham) before global cerebral ischemias were not induced. C and D are photographs of coronal slices of dorsal hippocampus of rats of control groups administered saline for 7 days, 10 minutes after global cerebral ischemias were induced. E and F are photographs of coronal slices of dorsal hippocampus of rats of groups administered composition of YKD solution for 7 days, 10 minutes after global cerebral ischemias were induced.

FIG. 2 is a graph showing changes of body temperatures administered YKD solution, after inducement of global cerebral ischemia and reperfusion.

FIG. 3 is a graph showing results of numbers of neurons countered at regions of brain hippocampuses of rats of normal group (sham) (In normal group global cerebral ischemias were induced.), saline group (control) and YKD group, 7days after global cerebral ischemias were induced.

FIG. 4 is a graph showing passive avoidance learning power administered with YKD.

FIG. 5 is a graph showing water maze test.

### DETAILED DESCRIPTION OF THE INVENTION

The present inventors carried out an intensive study and found out the fact that the present composition blocks necrosis of neurons.

Accordingly, an object of the present invention is to provide a composition of comprising 5˜20 weight parts of Astragali Radix, 5˜10 weight parts of Atractyodis Rhizoma, 5˜10 weight parts of Notopterygii Rhizoma, 5˜10 weight parts of Langanae Cortex, 5˜10 weight parts of Lycii Leaf or Fructus, 2˜10 weight parts of Cnidium Rhizoma, 2˜10 weight parts of Glycyrrhizae Radix, 2˜10 weight parts of Horn of Cervi Parvum, 5˜10 weight parts of Polygoni Multiflori Radix and 2˜10 weight parts of Paeonia Radix as main ingredients.

The present invention is further to provide a composition comprising one or more ingredient(s) selected from the group consisting of Ginseng Radix, Angelicae Gigantis Radix, Cornus Fructus, Dioscoreae Rhizoma, Rehmanniae Radix, Cuscutae Semen, Shizandra Semen, Rubi Fructus, Puerariae Radix, Artemisiae Capillaris Herba, Zizyphi semen, Zingiberis Rhizoma, Cinnamomi Cortex and Muschus as auxiliary components in addition to the said main components. Each auxiliary ingredient can comprise 2˜10 weight parts (In the case of Muschus, 0.01˜1.0 weight part).

Each content of the main or auxiliary ingredient is based on the dried weight. Each ingredient of the main or auxiliary ingredient can be used as powdered state, or as extrat state extracted from water, alcohol or mixture thereof. Extract is much more convenient in handling, administration and storage.

The composition can be used as powder, tablet, pill, capsule, syrup, oral solution, solution for injection.

Generally, according to a theory of oriental medicine (natural products), even though similar oriental medicinal composition, anticipating effect or side effect is very different. Accordingly, in the present invention optimal composition is accomplished so as to enhance clinical effect for prevention and treatment of dementia and minimize side effect.

The present composition has excellent effect of inhibition of apoptosis of neuron due to cerebral ischemia. Therefore, the present composition can be used in prevention and treatment of brain disease.

The present composition has nearly not side effects and toxicities and has an improved pharmacological effects comparing with ordinary therapeutics for dementia until now.

Main ingredients of Astragali Radix are polysaccharide and saponin which are effects in promotion of antibody-formation. Astragali Radix has anti-aging effect, elongation of life of Drosophila, vascularization effect, lowering of blood pressure and aggregation of platelet.

Main ingredients of Atractyodis Rhizoma are essential oil such as β-eudesmol and hinesol. They have effects of sedation, anticonvulsion, depression of central nerve system, protection of liver action and antiulcer.

Notopterygii Rhizoma has about 2.3% of essential oil such as α-nopinen, β-nopinen, lionen and bormylacetate and has effect of analgesia against various pain.

Main ingredients of Langanae Cortex are fat, protein, soluble nitrogen compound, saccharide and organic acid. Langanae Cortex has effects of protections of heart and spleen, cleaning of blood and tranquilizing.

Main ingredients of Cnidii Fructus are vitamin B1, B2, C, nicotinic acid, β-sitosterol and linoleic acid. Cnidii Fructus has effects of protections of kidney and lung and liver and also effect of diabetes.

Main ingredients of Cnidium Rhizoma are cnidilide, ligustilide, neocnidilide, butylphthalide, sedanoic acid and essential oils. Cnidium Rhizoma has effects of harmonization of blood circulation and menstruation and analgesic,

Main ingredients of Glycyrrhizae Radix are saponin of triterpene series, glycyrrhizin and various glycosides of flavonoid series. Glycyrrhizae Radix is used as auxiliary agent in oriental prescriptions.

Main ingredients of Horn of Cervi Parvum are various aminoacids, amines, chondroitin sulfate, and various peptides which have effect of anti-inflammation. Horn of Cervi Parvum has effects of enhancing of protein synthesis, neucleic acid synthesis, hematopoietisis, raising of testosterone level in serum, enhancing of rate of work, enhancing of learning rate in mice, anti-aging and suppress of peroxidation of lipid.

Polygoni Multiflori Radix has effects of protection of liver, kidney, increase of myeolooiuesis stem cell, enhancing of immune.

Main ingredients of Paeonia Radix are paeoniplorine, paeoniol, paeonione, benzoic acid, essential oil, resin, tannin and starch. Paeonia Radix has effects of hematic, protection of liver, sudorrhea and harmonization of menstruation.

There is no report that any composition comprising the said main substances has effect of protection of neuron.

Main ingredients of Ginseng Radix, one of auxiliary substances are mixture of saponins of 13 kinds or more such as ginseng saponin, panaxatriol, panaxadiol, propanaxatriol, propanaxadiol, and essential oils, etc. Ginseng Radix has effects of tonic, anti-oxidation, anticancer, and other various pharmacological effects.

Main ingredients of Angelicae Gigantis Radix, one of auxiliary substances are various essential oils, soluble substances, and 23kinds of metal elements. Angelicae Gigantis Radix has effects of increasing of blood flow in coronary artery, anti-dysrhythmia, promotion of production of IL-2 and IgG immunes, analgesics, suppression of central nerve, anticancer, antibacteria, protection of liver, harmonization of menstruation. In addition, Volatile ingredients of Angelicae Gigantis Radix increases blood pressure, whereas soluble substances of Angelicae Gigantis Radix lowers blood pressure.

Cornus Fructus, one of auxiliary substances has effects of protection of liver and stomach, in high blood pressure, improving of dysfunction of endocrine, lowering of blood sugar level. And Cornus Fructus enhances IgG level in serum.

Dioscoreae Rhizoma, one of auxiliary substances has effects of protection of spleen, lung and kidney.

Rehmanniae Radix, one of auxiliary substances has effects of hematic, protection of kidney, tonic, suppression of evolution of T-cell and B-cell in serum due to synthesis of DNA of serum cell. Rehmanniae Radix has also effects of anti-aging, enhancing of activities of glutathione peroxidase and SOD in serum, raising aldosteron level in blood and lowering slowly body weight.

Cuscutae Semen, one of auxiliary substances has glycoside of resin-like and substances of vitamin-like and has effects of protection of liver and is also used in pollution.

Main ingredients of Shizandra Semen, one of auxiliary substances are essential oils of various kinds, sesquicaren, β-bisabolene, β-camigrene, α-pinene, citral, malic acid, succinic acid, fructose and resin. Shizandra Semen has effects of tonic, treatment of hydropedesis, pollution, protection of kidney and lung.

Main ingredients of Rubi Fructus one of auxiliary substances are a large quantity of organic acid, vitamin c and carbohydrates. Rubi Fructus has effects of protection of kidney, pollution, treatment of oliguria and treatment of early ejaculation.

Main ingredients of Puerariae Radix, one of auxiliary substances are various kinds of isoflavone, β-sitosterol, etc. Puerariae Radix has effects of dry mouth, dibetes, diarrhea and is used in neck pain due to hypertension.

Main ingredients of Artemisiae Capillaris Herba, one of auxiliary substances are scoparone which is an ingredient of cholagogue, chlorogenic acid, β-pinene, etc., and unsaturated fatty acid. Artemisiae Capillaris Herba has effects of treatment of jaundice, diuresis, itching and hepatitis.

Main ingredients of Zizyphi semen, one of auxiliary substances are proteins, carbohydrates, organic acids, pectin, vitamin A, Bs, C, trace calcium, phosphorus and iron. Zizyphi semen has effects of protection of spleen, stomach, detoxication, etc.

Main ingredients of Zingiberis Rhizoma, one of auxiliary substances are zingiberol, zingiberene, phellandrene, camphene, citral, linalool, methylheptone, nonyl aldehyde, d-borneol, zingerone and zingiberone. Zingiberis Rhizoma has effects of antiemetic, solution of expectoration.

Main ingredients of Cinnamomi Cortex, one of auxiliary substances are various volatile essential oils, especially cinnamon aldehyde. Cinnamomi Cortex has effects of tonic, protection of apoplexy and edema.

Muschus, one of auxiliary substances is a dried secreta secreted in incense pouch of Moschus moschiferus parvipes. Main ingredients of Muschus are muskone, normuskone, 5α-androstan-3,17-dione, 5β-androstan-3,17-dione and other fragrant ingredients. Muschus has effects of arousal, hemagogue, anti-inflammation, analgesic and is used in palsy, birth trouble, etc.

The present invention is composed of a composition having an excellent neuro-protecting activity comprising of natural products such as 5˜20 weight parts of Astragali Radix, 5˜10 weight parts of Atractyodis Rhizoma, 5˜10 weight parts of Notopterygii Rhizoma, 5˜10 weight parts of Langanae Cortex, 5˜10 weight parts of Lycii Fructus, 2˜10 weight parts of Cnidium Rhizoma, 2˜10 weight parts of Glycyrrhizae Radix, 2˜10 weight parts of Horn of Cervi Parvum, 5˜10 weight parts of Polygoni Multiflori Radix, 2˜10 weight parts of Paeonia Radix.

The present invention is further to provide a composition having an excellent neuro-protecting activity comprising one or more components selected from the group consisting of Ginseng Radix, Angelicae Gigantis Radix, Cornus Fructus, Dioscoreae Rhizoma, Rehmanniae Radix, Cuscutae Semen, Shizandra Semen, Rubi Fructus, Puerariae Radix, Artemisiae Capillaris Herba, Zizyphi semen, Zingiberis Rhizoma, Cinnamomi Cortex and Muschus as auxiliary components in addition to the said main ingredients. Each auxiliary ingredient can comprise 2˜10 weight parts (In the case of Muschus, 0.01˜1.0 weight part).

The present composition can be used 100 mg 5000 mg/day based on a grown-up but the daily dose can be varied by sex, age, body weight and the degree of disease of patents.

FIG. 1 is photographs of cresyl violet-stained hippocampal regions of rats by microscope.

In FIG. 1, A and B are photographs of coronal slices of dorsal hippocampus of rats of control group (sham) before global cerebral ischemias were not induced.

C and D are photographs of coronal slices of dorsal hippocampus of rats of control groups administered saline for 7 days, 10 minutes after global cerebral ischemias were induced.

E and F are photographs of coronal slices of dorsal hippocampus of rats of groups administered composition of YKD solution for 7 days, 10 minutes after global cerebral ischemias were induced.

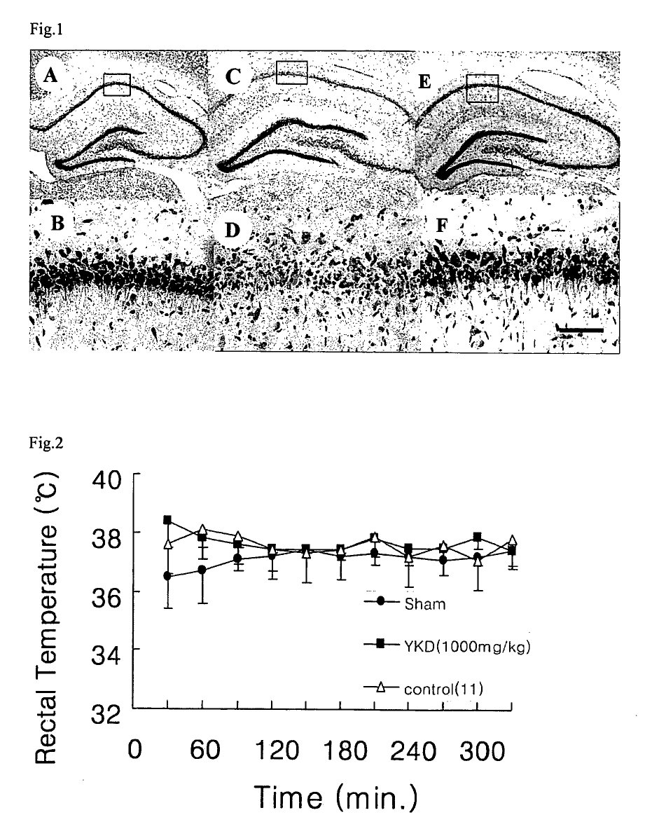

FIG. 2 is a graph showing changes of body temperatures administered YKD solution, after inducement of global cerebral ischemia and reperfusion.

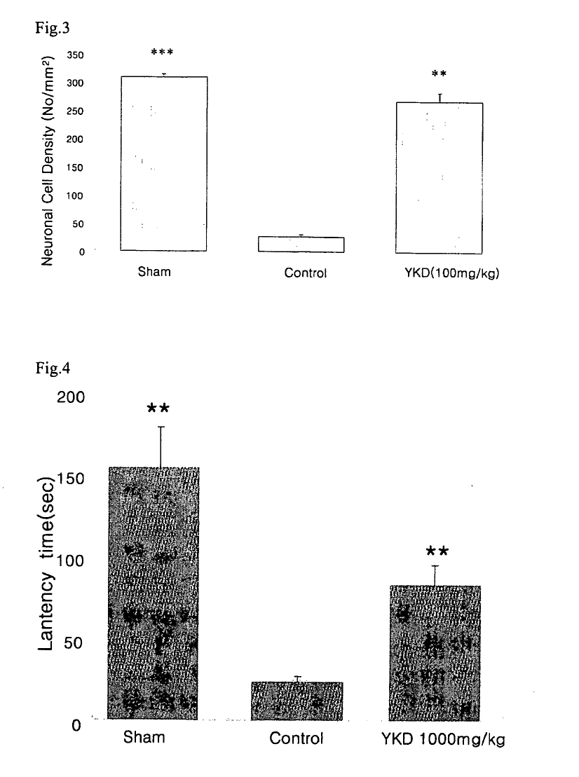

FIG. 3 is a graph showing results of numbers of neurons countered at CA1 regions of brain hippocampuses of rats of normal group (sham), saline group (control) and YKD group, 7days after global cerebral ischemias were induced.

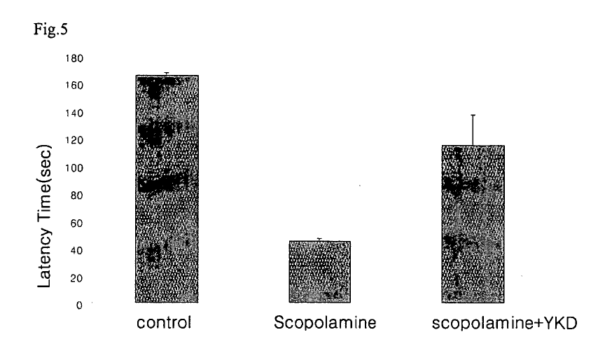

FIG. 4 is a graph showing passive avoidance learning power administered with YKD.

FIG. 5 is a graph showing water maze test.

The present invention will now be described in more detail in connection with the following examples and experimental examples which should be considered as being exemplary and not limiting the present invention.

### EXAMPLE 1

### Preparation of Water Extract

To 26.4 g of Astragali Radix, 17.6 g of Atractyodis Rhizoma, 17.6g of Notopterygii Rhizoma, 17.6 g of Langanae Cortex, 13.4 g of Cnidii Fructus, 8.8 g of Cnidium Rhizoma, 8.8g of Glycyrrhizae Radix, 8.8 g of Horn of Cervi Parvum, 17.6 g of Polygoni Multiflori Radix and 8.8 g of Paeonia Radix, 1 L of water was added and the mixture was heated slowly and refluxed for 6˜8 hours. The mixture was filtered with filter paper. The obtained filtrate was lyophilized to obtain 3 g of brown-colored powder.

### EXAMPLE 2

### Preparation of Alcohol Extract

The same mixture of ingredients of the example 1 was extracted with 1 L of ethanol for 3 hours. The mixture was filtered with filter paper. The obtained filtrate was lyophilized to obtain 3 g of brown-colored powder.

### EXAMPLE 3

### Preparation of 50% aqueous ethanol extract

The same mixture of ingredients of the example 1 was extracted with 1 L of 50% aqueous ethanol for 3 hours. The mixture was filtered with filter paper. The obtained filtrate was lyophilized to obtain 3 g of brown-colored powder.

### EXAMPLE 4

### Preparation of Water Extract

24 g of Astragali Radix, 15 g of Atractyodis Rhizoma, 15 g of Notopterygii Rhizoma, 15 g of Langanae Cortex, 10 g of Cnidii Fructus, 8.8 g of Cnidium Rhizoma, 8.8 g of Glycyrrhizae Radix, 8.8 g of Horn of Cervi Parvum, 17.6 g of Polygoni Multiflori Radix and 8.8 g of Paeonia Radix, 8.8 g of Ginseng Radix, 5.0 g of Angelicae Gigantis Radix and 8.8 g of Cornus Fructus were added to 1 L of water. The mixture was heated slowly and refluxed for 6˜8 hours. The mixture was filtered with filter paper. The obtained filtrate was lyophilized to obtain 3 g of brown-colored powder.

### EXAMPLE 5

24 g of Astragali Radix, 15 g of Atractyodis Rhizoma, 15 g of Notopterygii Rhizoma, 15 g of Langanae Cortex, 10 g of Cnidii Fructus, 8.8 g of Cnidium Rhizoma, 8.8 g of Glycyrrhizae Radix, 8.8 g of Horn of Cervi Parvum, 17.6 g of Polygoni Multiflori Radix and 8.8 g of Paeonia Radix, 8.8 g of Ginseng Radix, 5.0 g of Angelicae Gigantis Radix and 8.8 g of Cornus Fructus, 5.5 g of Rehmanniae Radix, 5.0 g of Cuscutae Semen and 5.0 g of Shizandra Semen were added to 1 L of water. The mixture was heated slowly and refluxed for 6˜8 hours. The mixture was filtered with filter paper. The obtained filtrate was lyophilized to obtain 4 g of brown-colored powder.

### EXAMPLE 6

### Preparation of 50% Aqueous Ethanol Extract

24 g of Astragali Radix, 15 g of Atractyodis Rhizoma, 15 g of Notopterygii Rhizoma, 15 g of Langanae Cortex, 10 g of Cnidii Fructus, 8.8 g of Cnidium Rhizoma, 8.8 g of Glycyrrhizae Radix, 8.8 g of Horn of Cervi Parvum, 17.6 g of Polygoni Multiflori Radix and 8.8 mg of Paeonia Radix, 8.8 g of Ginseng Radix, 5.0 g of Angelicae Gigantis Radix and 8.8 g of Cornus Fructus, 5.5 g of Rehmanniae Radix, 5.0 g of Cuscutae Semen and 5.0 g of Shizandra Semen were added to 1 L of aqueous ethanol. The mixture was heated slowly and refluxed for 6˜8 hours. The mixture was filtered with filter paper. The obtained filtrate was lyophilized to obtain about 4 g of brown-colored powder.

### EXAMPLE 7

24 g of Astragali Radix, 15 g of Atractyodis Rhizoma, 15 g of Notopterygii Rhizoma, 15 g of Langanae Cortex, 10 g of Cnidii Fructus, 8.8 g of Cnidium Rhizoma, 8.8 g of Glycyrrhizae Radix, 8.8 g of Horn of Cervi Parvum, 17.6 g of Polygoni Multiflori Radix and 8.8 g of Paeonia Radix, 8.8 g of Ginseng Radix, 5.0 g of Angelicae Gigantis Radix and 8.8 g of Cornus Fructus, 5.5 g of Rehmanniae Radix, 5.0 g of Cuscutae Semen, 5.0 g of Shizandra Semen, 5.0 g of Rubi Fructus, 5.0 g of Puerariae Radix, 5.0 g of Artemisiae Capillaris Herba, 5.0 g of Zizyphi semen, 5.0 g of Zingiberis Rhizoma and 5.0 g of Cinnamomi Cortex were added to 1 L of water. The mixture was heated slowly and refluxed for 6˜8 hours. The mixture was filtered with filter paper. The obtained filtrate was lyophilized to obtain about 4.5 g of brown-colored powder.

### EXAMPLE 8

24 g of Astragali Radix, 15 g of Atractyodis Rhizoma, 15 g of Notopterygii Rhizoma, 15 g of Langanae Cortex, 10 g of Cnidii Fructus, 8.8 g of Cnidium Rhizoma, 8.8 g of Glycyrrhizae Radix, 8.8 g of Horn of Cervi Parvum, 17.6 g of Polygoni Multiflori Radix, 8.8 g of Paeonia Radix, 5.0 g Rehmanniae Radix, 5.0 g of Cuscutae Semen, 5.0 g of Shizandra Semen, 5.0 g of Rubi Fructus, 5.0 g of Puerariae Radix, 5.0 g of Artemisiae Capillaris Herba, 5.0 g of Zizyphi semen, 5.0 g of Zingiberis Rhizoma, 5.0 g of Cinnamomi Cortex and 0.1 g of Muschus were added to 1 L of water. The mixture was heated slowly and refluxed for 6˜8 hours. The mixture was filtered with filter paper. The obtained filtrate was lyophilized to obtain about 4.3 g of brown-colored powder.

### Experimental Example 1

Test material: A lyophilized aqueous extract of the Example 1 was dissolved in normal saline to produce a concentration of 1000 mg/ml. (It refers to as Code No. “YKD”.)

Inducement of Cerebral Ischemia

Each 8 Male Wistar rats (SPF, Slc, Japan) aged 5 weeks and weighing about 170 g for each group was used for ischemia testing. The rats were adjusted at test environment with food and water freely available for 1 week.

Rats were anesthetized with 5% isoflurane (Baxter, USA) in a mixture of 70% nitrogen and 30% oxygen; anesthesia was maintained with 1.5% isoflurane. After anesthetization, a rat was laid on its back in a stereotaxic apparatus with the head held at a downward angle of 30° on the horizontal die of the apparatus while the nose and the mouth were fitted into a plastic cone connected to an anesthesia device (Ohrameda V.M.C./Boc Health Care, Cyprian, UK).

The tail was fixed on the operating table while the cervical vertebrae were extended. First the throat region was opened and silicon tube rings were inserted into the common carotid arteries. During inducement of ischemia, in order to block circulation of capillary vessel, in order to position trachea, esophagus, extermal jugular vein, common carotid arteries posterior, cervical and paravertebral muscle were passed through with thread and the wounds were sutured with operating clip.

Next, the rat was laid on its stomach and a micro electrocautery needle <1 mm in diameter was inserted through the alar foramina of the first cervical vertebra into the tunnel through which the vertebral artery runs. Both branches of the vertebral artery were occluded by electrical cauterization; operating clips were used as sutures. After 24 hours, after operating clip was removed, common carotid artery were ligated with aneurysm clip to induce ischemia for 5 min, 10 min, 20 min and 30 min. In case light reflex was not disappeared in 1 min, collum suture was ligated firmly, rats whose lignt reflex was not disappeared was excluded from the tests, because bilateral syntropic CA1 neuron injury was not induced, spasmodic rats were excluded too. To induce ischemia, the silicon tube rings were tightened so as to occlude the common carotid arteries. If the rat's light reflex vanished within 1 min, the cervical thread was tightened further. After 10 min of occlusion, the tube rings were loosened and the thread was removed, permitting reperfusion. Only those rats that were unconscious for 20±5 min after reperfusion were chosen for further study.

### Experimental Example 2

To evaluate histological valuation, one week after rats whose ischemia were induced, the rats were anesthetized, the rats were toracotomized and right ventricles were dissected. Niddles (No. 18) were inserted in left ventricles. Heparin-treated 5% sodium nitrite (Sigma, USA) saline was perfused in hearts. Subsequently, 4.0% paraformaldehyde fixation solution of pH 7.4 was perfused. After then, brains were removed, post-fixated in 4.0% paraformaldehyde fixation solution, immersed in 30% sucrose solution and fixed for one day at 4° C. Coronal blocks in dorsal hippocampuses between Bregma-2.5 mm and −4.0 mm from the fixed brains were prepared. After blocks were frozen, tissue segments were prepared. Tissue segments for sample preparation were collected for each 30 μm.

### Experimental Example 3

Segments including dorsal hippocampus were dyed and fixed with cresyl violet and at 1,000 m length of middle zones which were very apt to be damaged by delayed neuronal death among dorsal hippocampal CA1. The numbers of neurons were observed. Observation of the numbers of neurons were performed at high magnification (×250) by observing total 6 parts of 2 parts of right and felt of 3 different tissue segments from one brain tissue of pyramidal cells showing normal morphology with 3 observers and were averaged. The observers were not known the informations of test samples at the time of observation. The results were shown in FIG. 3.

### Experimental Example 4

The body temperature of each rat was monitored every 30 min for 6 hours after the induction of ischemia. In the case of descending of body temperature, the changes of body temperatures were measured with maintaining the descending state of body temperatures. And with preventing partly descending of body temperatures, the body temperatures were measured with preventing of defending effects of neurons. The two tests were carried out separately. Heat lamps were used to maintain body temperature at 37±5° C. during induction of ischemia, reperfusion, and recovery. To estimate brain temperature, body temperature was measured with a probe inserted at least 6 cm into the rectum (Miyazawa & Hossmann, 1992). YKD (100 mg/kg, i.p.) was administered to rats 0 and 90 min after induction of ischemia. Ischemia-only animals were injected i.p. with 180 μl/kg distilled water at the same time points. Beginning the day after ischemia induction, some animals were administered 200 μl YKD. The results were shown in FIG. 2 graph. As shown in FIG. 2, in the groups which YKD was administered, descending of body temperature was not appeared.

### Experimental Example 5

Water Maze Testing

Spatial memory tests were performed as described (Morris, 1984) with slight modification. A round pool (for rats, mice: diameter, 186 cm, 100 cm; height, 60 cm, 30 cm), was filled to a height of 10 cm below the tank rim with 22±1° C. water made opaque with powdered milk. The pool was situated in a dimly lit room and surrounded by four uniformly distributed visual cues for orientation. A plastic platform 10 cm in diameter was placed 1 cm below the water surface, midway between the center and rim of the pool in one quadrant. A video tracking system (Noldus, the Netherlands) was used to record animal location and to determine total swim latency (time to reach the platform), distance, and speed. An efficiency ratio was calculated by dividing the animal's swim distance by the straight-line distance from the entry point to the platform. Animals from the various experimental groups were tested in a random order that was repeated on each day of testing; testing was performed from 10 μm to 3 μm, corresponding to the animals' active period. Working memory testing, the acquisition trials, lasted for five consecutive days, with a two-session trial given on each of days 2.5. Day 1 consisted of 90 s swimming in the presence of the platform. Twice on each of the next four days, animals were tested in the presence of the platform in two identical sessions (Sessions 1 and 2), conducted as follows. An animal was placed in the water near the perimeter of one quadrant, but released facing a direction that varied with each session. Once the animal located and climbed on the platform, it was allowed to rest for 30 s. If, after 90 s, an animal had not located the platform, the experimenter would place it on the platform for 30 s. The second session was begun 20 min after the end of the first. The platform location was changed on each subsequent day. For the probe trial on day 6, the platform was removed and the animal was released as before. The decrease in session 1 escape latency from one day to the next, and the probe trial latency, represent improvement in long-term memory, whereas that from session 1 to session 2 measures improvement in working or short-term memory (Morris, 1983). Rats, ischemia groups, received either YKD extract (100 mg/kg, 200 μl, p.o.) or 200 μl saline p.o. daily during the test period.

### Experimental Example 6

Passive Avoidance Behavior

A fundamental study on learned abiotic theory was carried out by Richter (1957). That is, learned abiotics can not demonstrate on any results which a organism reacts and appears because any result which will happen can not be controled or avoided. The apparatus of this test has partition having guillotine door and at the bottom, a electrically conductive stainless grid was floored. The box (the apparatus) divided to 2 rooms by using partition door. Each room is under of noise of 60 dB. The test was performed at dimly-lit rooms. Among 2 rooms partitioned, in one room, mice were placed and by rendering electric shock, noise and lightening, the partition was opened. Then, the mice examined here and there and went into the other room which had no electric shock, no light and no noise. Then the door automatically closed. By measuring the time from opening of the partition to closing of the partition, latency time was measured. The test was carried out once a day for 1 week. The latency times of normal group (sham), YKD group and control group were compared. YKD group was administered 1000 mg/kg p.o. The results were shown in FIG. 4.

### Experimental Example 7

Analysis of Acetylcholinesterase

In the brain of dementia patent, it shows that loss of neuron and specially loss of neuron which is essential in memory and awareness is severe. Nerve tissue bundle and encephalolith in cerebral cortex are appeared. In dementia patent, 50% of loss of acetylcholine is appeared. Acetylcholinestrase is an enzyme which degrades acetylcholine into choline and acetic acid, which is the neurotransmitter in central and peripheral nerve systems and performs an important role in memory and learning activity. Acetylcholinesterase is under developing as therapeutic agent for dementia and AchE inhibitor. The inhibition rate of AchE showed effect of 64% at the concentration of 100 mg/kg.

As shown from the above experiments, the present composition was confirmed having an excellent neuro-protecting activity.

1. A composition having an excellent neuro-protecting activity comprising of natural products of 5˜20 weight parts of Astragali Radix, 5˜10 weight parts of Atractyodis Rhizoma, 5˜10 weight parts of Notopterygii Rhizoma, 5˜10 weight parts of Langanae Cortex, 5˜10 weight parts of Lycii Fructus, 2˜10 weight parts of Cnidium Rhizoma, 2˜10 weight parts of Glycyrrhizae Radix, 2˜10 weight parts of Horn of Cervi Parvum, 5˜10 weight parts of Polygoni Multiflori Radix, 2˜10 weight parts of Paeonia Radix of which substances are powdered or extracted with water, alcohol or mixture thereof.

2. A composition having an excellent neuro-protecting activity, in addition to the main substances of the claim 1, further comprising of one or more substances as auxiliary substance(s) selected from the group consisting of Ginseng Radix, Angelicae Gigantis Radix, Cornus Fructus, Dioscoreae Rhizoma, Rehmanniae Radix, Cuscutae Semen, Shizandra Semen, Rubi Fructus, Puerariae Radix, Artemisiae Capillaris Herba, Zizyphi semen, Zingiberis Rhizoma, Cinnamomi Cortex and Muschus of which substances are powdered or extracted with water, alcohol or mixture thereof and of which each substances are contained in 2˜10 weight parts (in the case of Muschus 0.01˜1.0 weight parts).

3. A formulation of composition of the claim 1 or 2 formulated in the form of powder, tablet, pill, capsule, syrup, oral solution, solution for injection.

4. A process for the preparation of a composition having an excellent neuro-protecting activity characterized in that of 5˜20 weight parts of Astragali Radix, 5˜10 weight parts of Atractyodis Rhizoma, 5˜10 weight parts of Notopterygii Rhizoma, 5˜10 weight parts of Langanae Cortex, 5˜10 weight parts of Lycii Fructus, 2˜10 weight parts of Cnidium Rhizoma, 2˜10 weight parts of Glycyrrhizae Radix, 2˜10 weight parts of Horn of Cervi Parvum, 5˜10 weight parts of Polygoni Multiflori Radix, 2˜10 weight parts of Paeonia Radix are powdered or extracted with water, alcohol or mixture thereof.

5. A process for the preparation of a composition having an excellent neuro-protecting activity characterized in that substances of claim 1 and one or more substances as auxiliary substance(s) selected from the group consisting of Ginseng Radix, Angelicae Gigantis Radix, Comus Fructus, Dioscoreae Rhizoma, Rehmanniae Radix, Cuscutae Semen, Shizandra Semen, Rubi Fructus, Puerariae Radix, Artemisiae Capillaris Herba, Zizyphi semen, Zingiberis Rhizoma, Cinnamomi Cortex and Muschus (Each content(s) of each auxiliary substance(s) is(are) 2˜10 weight parts, in the case of Muschus is 0.01˜1.0 weight part) are powdered or extracted with water, alcohol or mixture thereof.
